(19) United States
(12) Patent Application Publication
(10) Pub. No.: US 2024/0261786 A1
(43) Pub. Date: Aug. 8, 2024

(54) PRODUCT FOR PROVIDING MEMBRANE ELEMENTS AND MANUFACTURING METHOD

(71) Applicant: Sartorius Stedim Biotech GmbH, Göttingen (DE)

(72) Inventors: Alfons Kesting, Göttingen (DE); Wei Sun, Dassel (DE); Marcel Bremerich, Göttingen (DE); Louis Villain, Hannover (DE)

(73) Assignee: Sartorius Stedim Biotech GmbH, Göttingen (DE)

(21) Appl. No.: 18/566,208

(22) PCT Filed: May 23, 2022
(86) PCT No.: PCT/EP2022/063842

§ 371 Date: Dec. 1, 2023

(30) Foreign Application Priority Data
Jun. 11, 2021 (DE) 10 2021 003 009.5

(51) Int. Cl. B01L 3/00; B32B 3/30; B32B 7/06; B32B 7/12; B32B 23/06; B32B 23/08; B32B 23/20; B32B 27/08; B32B 27/10; B32B 27/28; B32B 37/12; B32B 37/18; B32B 38/10

(52) U.S. Cl. CPC B01L 3/502761; B32B 3/30; B32B 7/06; B32B 7/12; B32B 23/06; B32B 23/08; B32B 23/20; B32B 27/08; B32B 27/10; B32B 27/286; B32B 37/12; B32B 37/182; B32B 38/10; B01L 2200/0647; B01L 2200/12; B01L 2300/0681; B01L 2300/0851; B01L 2300/087; B32B 2250/03; B32B 2255/12; B32B 2255/26; B32B 2307/726; B32B 2307/7376; B32B 2307/748; B32B 2310/0843

(57) ABSTRACT

The invention relates to a product for providing membrane elements, comprising: a carrier film; and a membrane film, at least comprising: a support layer; and a membrane layer; the membrane film is releasably coupled to the carrier film; and the membrane film has a plurality of substantially discrete membrane elements. The invention also relates to a corresponding manufacturing process.

### TECHNICAL FIELD

The invention relates to a product for providing membrane elements and to a method for producing such a product.

### BACKGROUND

Test methods that use a membrane material with fixed DNA, RNA, and/or fixed proteins are often used to detect certain substances, in particular antibodies, in a sample. This molecular biological method is used in particular in laboratory medicine and pharmaceutical research, for example as a “Lateral Flow Assay” or test strip.

The membrane material can also be “functionalized”, i.e., have a surface structure that influences and/or controls the behavior, in particular a microfluidic behavior, of the sample. In this manner, for example, the reaction intensity of the samples can be increased.

To analyze the tests, the membranes are inserted into an analyzer following the application of a sample. This requires cutting the membranes into a suitable shape to securely and accurately position the membranes in the analyzer.

However, cutting the membranes to shape increases the workload and reduces test capacity. Because the membrane can shift out of position and/or jam, it is also often not possible to verify that the membrane is securely inserted into an analyzer and/or a test cartridge.

### SUMMARY

In light of the identified deficits, an object of the present invention is to provide a product for the improved provision of membrane elements and a method to manufacture such a product.

This problem is solved by the subject-matter of the independent claims. Advantageous embodiments are defined in the dependent claims.

A first aspect of the invention relates to a product for providing membrane elements at least comprising: a carrier film; and a membrane film at least comprising: a support layer; and a membrane layer; the membrane film is releasably coupled to the carrier film; and the membrane film has a plurality of substantially discrete membrane elements.

The product is in particular adapted to provide a plurality (preferably 2 or more, further preferably more than 20, further preferably more than 100) of membrane elements, wherein the membrane elements can be individually released and/or decoupled from the product. The support layer of the membrane film is advantageously releasably coupled to the carrier film such that the membrane film can be released, in particular peeled off, from the carrier film.

Individual membrane elements are preferably adapted and/or formed and/or shaped such that they can be inserted substantially accurately into a test cartridge and/or into an analyzer and/or into a sample holder.

Particularly preferably, the product has an elongated and/or oblong and/or tape-like shape and is further preferably adapted to be unwound from a roll. This allows space-saving storage and advantageous provisioning of the membrane elements, for example for use in an analysis method.

The support layer preferably comprises and/or consists of the following: PET (polyethylene terephthalate), PE (polyethylene), EVA (ethylene-vinyl acetate copolymer), PP (polypropylene), PVC (polyvinyl chloride), EVOH (ethylene-vinyl alcohol copolymer), PA (polyamide), and/or a similar material, in particular a material that has a low thickness as well as a high degree of flexibility and durability.

The support layer advantageously has a thickness of about 10 μm to about 20 μm, in particular about 12 μm.

The support layer is advantageously arranged at least regionally between the carrier film and the membrane layer.

The membrane layer preferably comprises a porous, in particular a microporous, and/or an absorbent, in particular highly-absorbent, material.

A porous, absorbent material, for example, has a sponge structure, and is adapted to collect and/or absorb fluids. The membrane layer preferably comprises a microfilter membrane layer and/or the membrane layer has a pore size, in particular a nominal, medium, and/or maximum pore size, between about 0.1 μm and about 20 μm, preferably between about 5 μm and 15 μm, which in particular generates an advantageous capillary effect of the membrane layer for transporting fluids on and/or in and/or along the membrane layer.

The membrane layer advantageously comprises and/or consists of the following: Cellulose, in particular cellulose nitrate (nitrocellulose) and/or cellulose acetate, and/or polysulfone, in particular polyether sulfone (PESU), dextran polymers and/or a material with similar characteristics and/or similar properties, in particular substantially sponge-like materials and/or materials that permit a lateral flow of fluids. In particular, a material with a high protein-binding capacity is advantageous for the aforementioned analysis methods.

The membrane layer advantageously has a thickness of about 50 μm to about 200 μm.

The carrier film preferably comprises a carrier material having a silicone coating, more preferably a siliconized paper.

A silicone coating or a similar coating in particular facilitates the advantageous release of the carrier film from the support layer, in particular an essentially non-destructive release of the membrane film from the carrier material.

The carrier film advantageously has a thickness of about 100 μm to about 300 μm, further preferably about 120 μm to about 200 μm, in particular about 150 μm.

The support layer of the membrane film is preferably releasably coupled to the carrier film by means of adhesive.

In the sense of this application, releasably coupled substantially refers to the capacity of releasing without the influence of heat and/or solvents. In particular, releasably coupled describes the capacity of substantially non-destructively separating two layers that are releasably coupled to one another, for example by using tweezers or a similar tool. For example, a stack of post-it notes consists of a plurality of releasably coupled note sheets.

The membrane layer of the membrane film is further preferably coupled to the support layer of the membrane film by means of adhesive. In contrast to the substantially releasable coupling of the support layer to the carrier film, the coupling of the membrane layer to the support layer in particular exhibits increased strength and/or durability. In other words: The bond and/or coupling of the membrane layer to the support layer is preferably more resistant than the bond and/or coupling of the support layer to the carrier film, preferably by a factor of 5, further preferably by a factor of 10 or higher. The coupling between the membrane layer and the support layer is further preferred to be substantially unreleasable, i.e., a substantially non-destructive separation of the membrane layer and the support layer and/or detachment of a layer of the membrane film is not possible.

This is advantageous because when releasing and/or decoupling a membrane element from the product, an accidental and/or undesired release and/or decoupling of the membrane layer from the support layer is prevented, but at least made more difficult. This is preferably achieved by a lower adhesion of the adhesive to the carrier film compared to the support layer and/or membrane layer. Alternatively and/or additionally, different adhesives can be used, wherein the adhesive between membrane layer and support layer leads to a stronger bond than the adhesive between support layer and carrier film. Alternatively and/or additionally, the carrier film can have a release agent and/or a coating that reduces the adhesion of the adhesive, such as silicone.

The adhesive preferably comprises a contact adhesive and/or melt adhesive for releasably coupling the support layer to the carrier film and/or for coupling the membrane layer to the support layer.

Adhesives are generally essentially permanently-adhesive, and adhesive-bonded parts coupled and/or bonded with them can substantially be decoupled and/or separated from one another residue-free and/or non-destructively. A dispersion adhesive and/or a melt adhesive is particularly preferred. The adhesive is advantageously at least slightly hydrophobic, i.e., water-repellent and/or does not mix with, and/or absorb, fluids, in particular water. This is in particular advantageous because any influence on the fluid to be tested is prevented, but at least reduced. In particular, the adhesive, especially the contact adhesive, is free of volatile surfactants and/or alcohol.

Melt adhesives in particular facilitate quick and reliable coupling and/or joining of adhesive-bonded parts. Melt adhesives generally also have high chemical resistance, therefore preventing, or at least reducing, an influence on the fluids applied onto the membrane elements.

The adhesive properties of certain melt adhesives can also be reduced by heat exposure, therefore achieving a substantially non-destructive release of the adhesive-bonded parts. Melt adhesive is advantageous in particular for bonding the support layer to the membrane layer because these layers do not necessarily have to be releasable and/or uncouplable from one another.

The adhesive further preferably has a layer thickness of about 8 μm to about 20 μm, preferably of about 9 μm to about 14 μm, in particular about 10 μm.

The releasable and/or uncouplable membrane elements of the product preferably each comprise at least one region of the membrane layer, a corresponding and/or coupled region of the support layer, as well as at least a part of the adhesive provided for coupling these regions of the membrane layer to the support layer.

The membrane elements further preferably comprise at least a part of the adhesive provided for coupling the support layer to the carrier film. This is in particular advantageous because this permits a releasable coupling and/or gluing-in of the membrane elements into an analyzer to achieve a secure seat of the membrane elements.

Advantageously, a membrane element is spaced apart along at least a part of its outer circumference from one or more other membrane elements and/or from at least another region of the membrane film of the product. In other words: The membrane elements are preferably substantially discrete.

The membrane elements are preferably formed by ablating and/or cutting through and/or melting an area of the membrane layer and the support layer. Furthermore, a membrane element has on its outer circumference a substantially continuous and/or uninterrupted gap to an adjacent membrane element and/or to the membrane film of the product surrounding the membrane element.

Alternatively and/or additionally, the outer circumference of a membrane element can be at least regionally perforated, i.e., provided with a plurality of holes, which also permits simple release and/or uncoupling of the membrane element from the product.

In this context, it is particularly advantageous to define and/or form and/or shape the membrane elements by means of laser cutting, i.e., to ablate and/or cut through and/or melt the membrane layer and the support layer with laser radiation. Depending on the material and/or shape of the membrane elements, the power, wavelength, pulse energy, and/or pulse duration of the laser radiation can be adjusted accordingly. A machining and/or chemical method can be used alternatively and/or additionally.

For example, the membrane elements are formed and/or shaped and/or defined such that they have one or more flow lines, for example by removing the membrane layer and the support layer in a central region of the membrane element such that the lateral regions of a membrane element are separated from one another.

Alternatively and/or additionally, the membrane elements advantageously have an at least partially structured surface that was preferably produced by at least partially and/or regionally ablating the membrane layer. In particular, the membrane elements can have one or more structures in the membrane layer. A structure preferably forms one or more flow lines, in particular for guiding a fluid and/or controlling a fluid flow. As a result, a fluid can be advantageously guided onto and/or in and/or along the membrane layer.

In particular, different fluid paths can be substantially delimited from one another, in particular to prevent, but at least reduce, any influence between the fluid paths. A plurality of flow lines is advantageously adapted to be substantially uniform, i.e., isomorphic, such that fluids can be tested under substantially identical conditions.

The shape of the membrane elements can for example be formed by ablating and/or cutting through the membrane film, consisting of at least the membrane layer and the support layer. Alternatively and/or additionally, one or more structures in the membrane layer of the membrane elements can be provided and/or adapted, for example by ablating and/or cutting through at least a part of the thickness of the membrane layer.

In this context, it is in particular advantageous to ablate and/or cut through and/or melt by means of laser cutting, i.e., ablating and/or cutting through and/or melting by energy by means of laser radiation. Depending on the material and/or the shape and/or structure to be produced, the power, wavelength, pulse energy, and/or pulse duration of the laser radiation can be adjusted accordingly.

Particularly preferably, a plurality of membrane elements has substantially identical shapes and/or structures. Alternatively and/or additionally, one or more membrane elements can have deviating shapes and/or structures. For example, it is advantageous if the product provides different membrane elements, wherein the different membrane elements are for example required in consecutive process steps and/or are inserted into different test cartridges or analyzers. In particular, different membrane elements can be arranged in groups and/or in a repeating arrangement in and/or on the product.

Alternatively and/or additionally, a membrane element advantageously comprises one or more mixing and/or reaction zones and/or one or more application zones, which are preferably formed by one or more structures.

In particular, in a mixing and/or reaction zone, two or more fluids can be combined to be mixed and/or react with one another. A mixing and/or reaction zone can further comprise a substance that reacts with one or more fluids. Embedding and/or arranging and/or fixing DNA, RNA, and/or proteins (for example, by means of “dispensers” and/or “spötter”) is particularly preferred. Alternatively, a substance can be introduced into a mixing and/or reaction zone by other means, for example by applying droplets with a pipette.

An application zone can in particular be a region on the membrane layer into which a fluid is applied, wherein the fluid is preferably distributed from the application zone at least regionally in the membrane layer and/or across the surface of the membrane element (lateral flow).

The membrane elements can comprise substantially straight and/or at least regionally curved and/or arc-shaped lines for guiding a fluid. Different structures can be provided and/or regions of the membrane elements can be formed and/or adapted in accordance with requirements, for example a defined flow velocity of a fluid along a flow line. The formed and/or shaped flow lines and/or structures can in particular influence and/or control the microfluidic behavior of fluids on and/or in the membrane layer.

The product can further comprise an essentially continuous perforation, by which, for example, an advantageous provision of the membrane elements can be achieved by conveying the product in a dispenser. Alternatively and/or additionally, a corresponding mating part can be present in a test cartridge and/or in an analyzer, which permits accurate positioning and/or insertion.

A further aspect relates to a method for producing a product for providing membrane elements, in particular a product according to the first aspect of the present invention, comprising the steps: Provide a support layer; releasably couple a carrier film to a first side of the support layer, in particular by means of a contact adhesive and/or melt adhesive; couple a membrane layer to a second side of the support layer, in particular by means of a contact adhesive and/or melt adhesive; form a plurality of membrane elements by at least regionally ablating the support layer and/or the membrane layer. Preferably, the membrane layer and the support layer are coupled to one another in a substantially non-releasable manner, i.e., the coupling can essentially not occur without at least partial destruction of at least one of the layers.

The manufacturing method preferably firstly comprises providing a support layer, for example a thin PET film, and coupling a carrier film, for example a siliconized paper, to a side of the support layer. The method further preferably comprises coupling a membrane layer, for example comprising nitrocellulose, to the opposite side of the support layer. The method further preferably comprises subsequently forming a plurality of membrane elements in the membrane film (membrane layer and support layer). The membrane elements are preferably formed by ablating and/or cutting through and/or melting an area of the membrane layer and the support layer.

In this context, it is particularly advantageous to define and/or form and/or shape the membrane elements by means of laser cutting, i.e., to ablate and/or cut through and/or melt a region of the membrane layer and a corresponding region of the support layer with laser radiation. Depending on the material and/or shape of the membrane elements, the power, wavelength, pulse energy, and/or pulse duration can be adjusted accordingly. A machining and/or chemical method can be used alternatively and/or additionally.

Alternatively, the method can comprise the following steps: Form a membrane film, comprising the steps: Provide a support layer and a membrane layer; and couple the support layer to the membrane layer; releasably couple the support layer of the membrane film to a carrier film; form a plurality of membrane elements of the membrane film by at least regionally ablating the support layer and the membrane layer (membrane film).

The sequence of the process steps can vary. The membrane elements are preferably formed and/or shaped after the membrane layer, the support layer and the carrier film were coupled such that the formed and/or shaped membrane elements are fixed and/or supported by the carrier film.

Advantageously, the method further comprises the step: Structure the membrane elements by ablating at least a portion of the membrane layer of the membrane elements and/or at least a region of the membrane film.

At least a portion of the membrane elements can in particular be structured before the membrane layer is coupled to the support layer. This has the advantage that the integrity of the support layer is not influenced by the structuring.

Alternatively and/or additionally, the structuring can occur after the membrane layer is coupled to the carrier film. This has the advantage that the membrane film is additionally reinforced and/or stabilized by the carrier film.

The membrane elements are advantageously formed and/or shaped and/or structured by means of laser cutting.

Advantageously, the method further comprises one or more of the steps:
apply an adhesive to the support layer and/or to the membrane layer to couple the support layer to the membrane layer, preferably substantially unreleasably; and/or
apply an adhesive to the support layer and/or the carrier film to releasably couple the support layer to the carrier film.

The strength and/or durability and/or resistance strength of the coupling between the membrane layer and the support layer is preferably greater than between the support layer and the carrier film, in particular by a factor greater than 5. In other words: a greater force is required to separate and/or release the membrane layer from the support layer than to separate and/or release the support layer from the carrier film, in particular by a factor greater than 5.

Adhesive is preferably applied at least regionally to one or more sides of the support layer and/or the membrane layer and/or the carrier film, preferably with a layer thickness of about 8 μm to about 20 μm, in particular about 10 μm. The adhesive advantageously comprises a contact adhesive and/or a melt adhesive. The adhesive is advantageously sprayed on and/or brushed on and/or applied substantially uniformly by other means.

With reference to the figures, the following describes examples of individual embodiments for solving the task. In certain cases, the described individual embodiments have features that are not mandatory to execute the claimed scope, but provide desired properties in certain applications. Embodiments that do not have all the features of the embodiments described below are therefore also considered to be disclosed as falling under the described technical teaching. Furthermore, in order to avoid unnecessary repetitions, certain features are only mentioned in relation to individual embodiments described below. We note that the individual embodiments must therefore not only be reviewed in isolation, but also based on a combined review. Based on this combined review, the person skilled in the art will recognize that individual embodiments can also be modified by including individual or several features of other embodiments.

We note that a systematic combination of the individual embodiments with individual or several features described with reference to other embodiments can be desirable and expedient and must therefore be considered and must also be deemed to be included in the description.

### BRIEF DESCRIPTION OF THE DRAWINGS

FIG. 1 shows an exemplary embodiment of a product for providing a plurality of membrane elements, wherein a membrane element was detached from the product;

FIG. 2 shows an exemplary embodiment of the product for providing a plurality of membrane elements, wherein a portion of the membrane film was released from the carrier film of the product;

FIG. 3 shows exemplary structures 16 and gaps 24 as well as an exemplary arrangement of the elements of a preferred embodiment of the product 1;

FIG. 4a shows an exemplary method for producing a product for providing a plurality of membrane elements;

FIG. 4b shows an alternative, exemplary method for producing a product for providing a plurality of membrane elements;

FIG. 5 shows membrane elements with exemplary structures provided by a product;

FIG. 6 shows additional, exemplary membrane elements;

FIG. 7 shows a detail of an exemplary, structured membrane element.

### DETAILED DESCRIPTION

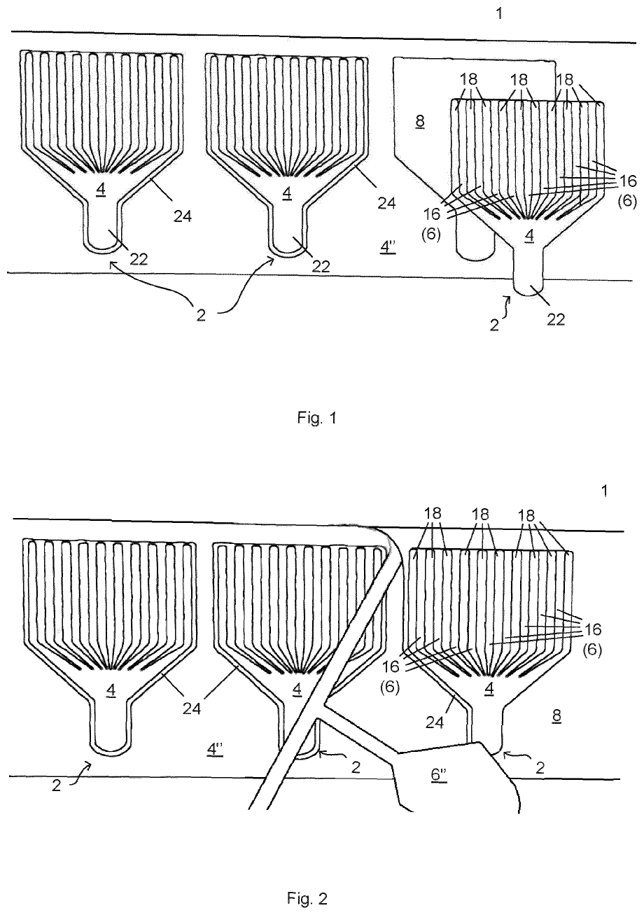

FIG. 1 shows an exemplary embodiment of a product 1 for providing a plurality of membrane elements 2, wherein one of the membrane elements 2 shown was released and/or uncoupled from the product 1. The membrane elements 2 are in particular adapted to be inserted into an analyzer and/or a provided section of a test cartridge.

FIG. 1 shows a section of a product 1 that has an essentially elongated and/or tape-like shape. The product 1 preferably comprises at least one carrier film 8 and a (preferably multilayered) membrane film that comprises at least one support layer 6 and a membrane layer 4.

The membrane layer 4 comprises a porous, in particular a microporous, and/or an absorbent (in particular highly-absorbent) material, which in particular comprises a sponge structure and is suitable for absorbing and/or collecting fluids. Depending on the application, the membrane layer preferably has an, in particular nominal, mean and/or maximum, pore size of approximately 5 μm or approximately 10 μm, which in particular facilitates a capillary effect of the membrane layer 4 for conveying fluids on and/or in and/or along the membrane layer. The membrane layer 4 in particular has a thickness of approximately 50 μm to approximately 200 μm.

The membrane layer 4 advantageously comprises and/or consists of the following: Cellulose, in particular cellulose nitrate (nitrocellulose) and/or cellulose acetate, and/or polysulfone, in particular polyether sulfone (PESU), dextran polymers and/or a material with similar characteristics and/or similar properties, in particular substantially sponge-like materials and/or materials that permit a lateral flow of fluids.

The carrier film 8 comprises a carrier material having a silicone coating, such as siliconized paper, therefore allowing the carrier film 8 to be substantially easily released, in particular substantially non-destructively released, from the support layer 6. The carrier film preferably has a material thickness of about 100 μm to about 300 μm.

The support layer 6 preferably has a thickness of about 10 μm to about 20 μm and consists of, for example: PET (polyethylene terephthalate), PE (polyethylene), EVA (ethylene-vinyl acetate copolymer), PP (polypropylene), PVC (polyvinyl chloride), EVOH (ethylene-vinyl alcohol copolymer), PA (polyamide), and/or a similar material having high flexibility and durability.

The carrier film 8 and the membrane film are at least regionally arranged one above the other, wherein the support layer 6 of the membrane film is releasably coupled and/or connected to the carrier film 8. The membrane film is preferably releasably coupled to the carrier film 8 such that the membrane film, in particular the membrane elements 2, can be substantially non-destructively released or decoupled from the carrier film 8—for example with the aid of tweezers. In particular, the membrane film is releasably coupled to the carrier film 8 by means of an adhesive, which comprises a contact adhesive and/or a melt adhesive.

The shown section of the product 1 comprises a plurality (e.g., three) of substantially discretely formed membrane elements 2. In this example, the membrane elements 2 are essentially identically formed and/or shaped, i.e., they have a substantially identical shape. Alternatively and/or additionally, one or more membrane elements 2 can have deviating shapes.

In the embodiment shown, the membrane layer 4 of the membrane elements 2 is delimited by a gap 24 along its outer circumference from a membrane layer 4″, which is not comprised by a membrane element 2. A gap 24 can in particular be generated by regionally ablating and/or removing and/or melting at least the membrane film, i.e., the membrane layer 4 and the support layer 6. Laser cutting is particularly suitable for this purpose.

Furthermore, the depicted membrane elements 2 are structured substantially identically: The membrane elements 2 comprise one or more (e.g., ten) flow lines 18, which are formed in the membrane layer 4 of the membrane elements 2 and are suited to guide a fluid applied to at least one application zone 22 away from the latter. The flow lines 18 are defined and/or formed by one or more structures 16 and/or are preferably spaced apart from one another, wherein the one or more structures 16 are generated in particular by (at least partially) ablating and/or removing and/or melting the membrane layer 4, for example by laser cutting.

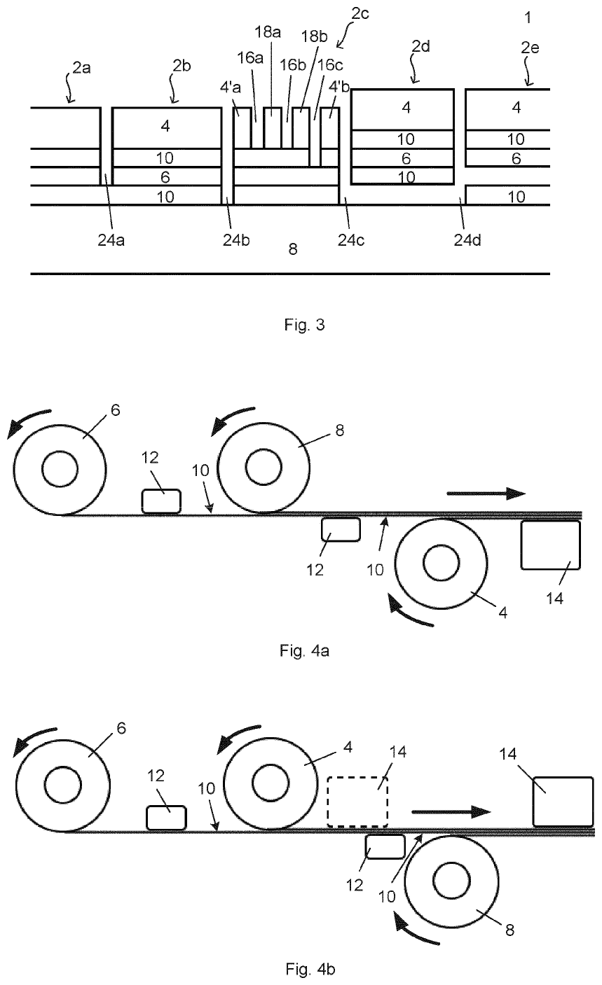

In the embodiment shown in FIG. 1, the membrane layer 4 and the adhesive 10 (see FIG. 3), which at least partially connects the membrane layer 4 to the support layer 6, was removed along the structures 16 such that the support layer 6 remains there and is in particular visible. This has the particular advantage that the shape of the membrane elements 2 is stabilized by the support layer 6 existing between (i.e., partially bridging) the flow lines 18 and/or substantially maintaining the structural stability of the membrane elements 2 after the latter are released from the product 1.

FIG. 2 shows an exemplary embodiment of the product 1 for providing a plurality of membrane elements 2, wherein the membrane layer 4″ and the support layer 6″ of the membrane film, which are not comprised by any membrane element 2 and/or which are not part of a membrane element 2, were released from the carrier film 8 of the product 1. On the product 1 shown, the membrane elements 2 are not formed over the entire surface of the membrane film of the product 1. The membrane layer 4″ and the support layer 6″ between the membrane elements 2 preferably have essentially the same features or properties as the membrane layer 4 or the support layer 6 of the membrane elements 2 respectively. In particular, the membrane layer 4″ and/or the support layer 6″ can also be separated (can be released or uncoupled), as shown in FIG. 2.

By pulling off and/or releasing and/or decoupling the membrane layer 4″ and support layer 6″ not comprised by a membrane element 2, the provision of membrane elements 2 can be improved because only the membrane elements 2 remain on the product 1. This allows the membrane elements 2 to be more readily identified and/or gripped and/or manipulated for detaching and/or decoupling the membrane elements 2 from the product 1, e.g., by means of tweezers.

FIG. 3 shows exemplary structures 16 and gaps 24 as well as an exemplary arrangement of the elements of a preferred embodiment of product 1.

The product 1 shown comprises at least one carrier film 8, which is releasably coupled to a support layer 6 by means of adhesive 10. The adhesive 10 preferably comprises a contact adhesive and/or a melt adhesive. The carrier film 8 has a material thickness of preferably about 100 μm to about 300 μm, e.g., about 150 μm. The carrier film 8 preferably comprises a carrier material having a silicone coating, more preferably a siliconized paper. Alternatively and/or additionally, the carrier film 8 can comprise another material, preferably a material with similar non-adhesive properties, thus achieving a releasable coupling and allowing a substantially non-destructive release of the support layer 6 from the carrier film 8a.

The support layer 6 has a material thickness of preferably about 10 μm to about 20 μm, e.g., about 12 μm. The support layer 6 preferably comprises and/or consists of the following: PET (polyethylene terephthalate), PE (polyethylene), EVA (ethylene-vinyl acetate copolymer), PP (polypropylene), PVC (polyvinyl chloride), EVOH (ethylene-vinyl alcohol copolymer), PA (polyamide), and/or a similar material. The support layer 6 is in particular used to support and/or stabilize the membrane elements 2 and/or the membrane layer 4.

The support layer 6 is preferably coupled and/or connected to the membrane layer 4 by adhesive 10, wherein the adhesive 10 is substantially identical to, or different from, the adhesive 10 provided between the support layer 6 and the carrier film 8. The thickness of the different adhesive layers 10 can be substantially identical or different. The membrane layer 4 has a material thickness of preferably approximately 50 μm to approximately 200 μm. The membrane layer 4 preferably comprises a porous, in particular a microporous, and/or an absorbent, in particular highly-absorbent, material, such as: Cellulose, in particular cellulose nitrate (nitrocellulose) and/or cellulose acetate, and/or polysulfone, in particular polyether sulfone (PESU), dextran polymers and/or a material with similar characteristics and/or similar properties.

Preferably, the adhesive 10 comprises a contact adhesive and/or melt adhesive to releasably couple the support layer 6 to the carrier film 8 and/or to, preferably substantially unreleasably, couple the membrane layer 4 to the support layer 6. Furthermore, the coupling between the membrane layer 4 and the support layer 6 preferably has a greater adhesion and/or resistance strength compared to the coupling between the support layer 6 and the carrier film 8. Adhesives 10 that permit decoupling and/or separating coupled and/or connected adhesive-bonded parts from one another substantially without residue and/or non-destructively are also preferred. The adhesive 10 is advantageously at least slightly hydrophobic, i.e., water-repellent and/or does not mix with fluids, in particular water.

The adhesion of the coupling and/or connection between the support layer 6 and the membrane layer 4 is preferably greater and/or more resistant than between the support layer 6 and the carrier film 8. This can be achieved, for example, by correspondingly different adhesives 10 and/or by appropriately selected materials and/or a treatment of the membrane layer 4 and the carrier film 8.

In particular when using a siliconized paper as a carrier film 8, a comparatively low adhesion between the membrane film and the carrier film 8 can be achieved, thus achieving simple release of the membrane film from the carrier film 8.

The shown section of the product 1 of FIG. 3 comprises a plurality (e.g., five) of membrane elements 2 that are substantially discretely formed, i.e., are substantially delimited from one another. The membrane elements 2 are separated and/or spaced apart from one another by gaps 24a-24d. Each individual membrane element 2 can therefore be released or removed from the carrier film 8 independently of other membrane elements 2.

As shown, the gap 24a is formed differently than the gaps 24b-24d: The gap 24a extends through the entire thickness of the membrane film, i.e., through the membrane layer 4, the support layer 6 and the adhesive 10, which connects the membrane layer 4 and the support layer 6. The membrane element 2e can in particular be decoupled from the product 1 by forming a membrane element 2 with such a gap 24a. The membrane element 2e comprises the membrane film (in particular the membrane layer 4, the support layer 6 and the adhesive 10 located between them). This is particularly advantageous if the released membrane element 2e is essentially not intended to have an adhesive 10 on the outside of the support layer 6, for example to facilitate handling the membrane element 2e.

Alternatively, a gap 24b-24d can be created by ablating and/or removing and/or melting the membrane layer 4, the support layer 6 and the adhesive layer(s) 10, which connect the membrane layer 4 to the support layer 6 or the support layer 6 to the carrier film 8. This is in particular advantageous in order to obtain the released membrane element 2d, which comprises adhesive 10 at least regionally on a side opposite to the membrane layer 4. In other words: The membrane elements 2d released or decoupled in this manner have adhesive 10 at least regionally on the side released from the carrier film 8. Such a membrane element 2d is advantageous, for example to achieve a secure hold of the membrane element 2d in an analyzer.

A secure positioning of the membrane elements 2 in an analyzer and/or a test cartridge can in particular be achieved because the membrane element 2d can be glued to the side of the support layer 6 in the analysis device by the adhesive 10 and/or can be releasably coupled with said glue.

FIG. 3 further shows a cross-section of a membrane element 2c, which has an exemplary structured membrane layer 4. The membrane element 2c comprises in particular two flow lines 18a and 18b formed by structures 16a-16c in the membrane layer 4.

One or more structures 16 are preferably formed and/or generated by ablating and/or cutting and/or removing and/or melting the membrane layer 4 and, optionally, the adhesive layer 10 of the membrane film. Laser cutting is in particular suitable for this purpose, as explained in more detail with respect to FIGS. 4a and 4b.

The flow lines 18a and 18b are in particular adapted to conduct a fluid along and/or in the membrane layer 4. The membrane layer 4, which forms the flow lines 18a and 18b, is substantially separated and/or delimited from the respective adjacent membrane layers 4′a and 4′b of the membrane element 2c by the structures 16. In particular, the membrane layers 4′a and 4′b thus do not come into contact with the fluid guided by the flow lines 18a and 18b.

A structure 16 preferably extends at least over a part of the thickness of the membrane layer 4, further preferably over substantially the entire thickness of the membrane layer 4 (see structures 16a and 16b of the membrane element 2c). Alternatively, a structure 16 extends through the membrane layer 4 and at least through a part of the thickness of the adhesive layer 10, which connects the membrane layer 4 and the support layer 6 (see structure 16c).

FIG. 4a shows an exemplary method for producing a product for providing a plurality of membrane elements 2 or a device suitable for executing the method for producing the product. The support layer 6 is preferably provided first, for example from a roll. On one side of the support layer 6, an adhesive 10 of at least one adhesive device 12 is applied and/or sprayed on at least regionally, and/or one side of the support layer 6 is wetted at least regionally with adhesive 10. An application of an essentially uniform and/or continuous layer of adhesive 10 by the at least one adhesive device 12 is preferred.

Subsequently, the carrier film 8 is preferably unrolled from a roll and placed onto, and/or coupled and/or connected to, the side of the support layer 6 comprising the adhesive 10.

Furthermore, adhesive 10 is preferably applied to the side of the support layer 6 opposite to the carrier film 8. This is in particular the same adhesive 10 as provided between the support layer 6 and the carrier film 8. The membrane layer 4 is subsequently placed onto, or coupled or connected to, the support layer 6.

A plurality of membrane elements 2 is then preferably formed and/or shaped and/or structured, for example by one or more ablation devices 14. An ablation device 14 is in particular adapted to at least regionally ablate and/or remove at least a portion of the product 1, in particular the membrane film. In particular, the ablation device 14 is adapted to selectively form the gaps 24 shown in FIG. 3 in the membrane layer 4, the support layer 6 and the layer(s) adhesive 10. The ablation device 14 is in particular adapted to selectively form one or more structure(s) 16 in the membrane element 2 (in particular the membrane layer 4).

For this purpose, ablation device 14 can comprise one or more means for cutting and/or melting at least a part of the membrane layer 4 and/or the adhesive 10 and/or the support layer 6.

An ablation device 14 preferably comprises one or more means for laser cutting in order to shape and/or form the membrane elements 2 and/or to shape one or more structures 16 in at least the membrane layer 4 of the membrane elements 2. Alternatively and/or additionally, an ablation device 14 can comprise one or more drilling and/or milling tools for forming and/or structuring the membrane elements 2.

Advantageously, the production of the product 1 or the individual steps of the method is carried out substantially continuously and/or in parallel such that large quantities of the product 1 can be produced in a time-efficient manner. The support layer 6, the carrier film 8 and the membrane layer 4 are in particular most suitably fed and/or provisioned as shown in FIG. 4, i.e., unrolled from a roll and fed substantially continuously to the various process steps.

Optionally, the device can preferably be provided with one or more devices for tempering, supporting, pressing and/or checking the product 1 and/or its components (not shown), in particular to obtain or ensure improved product quality.

FIG. 4b shows an alternative, exemplary method for producing a product for providing a plurality of membrane elements. In comparison to the method shown in FIG. 4a, the membrane film is first produced by coupling the membrane layer 4 to the support layer 6 by means of adhesive 10. Subsequently, the carrier film 8 is releasably coupled to the support layer 6 of the membrane film by means of adhesive and fed to the ablation device 14 for forming and/or structuring the membrane elements 2. The releasable coupling allows simple and/or substantially non-destructive separation of the support layer 6 from the carrier film 8.

Alternatively and/or additionally, at least one ablation device 14 can be arranged and/or positioned such that it structures the membrane layer 4 before the carrier film 8 is supplied and/or coupled.

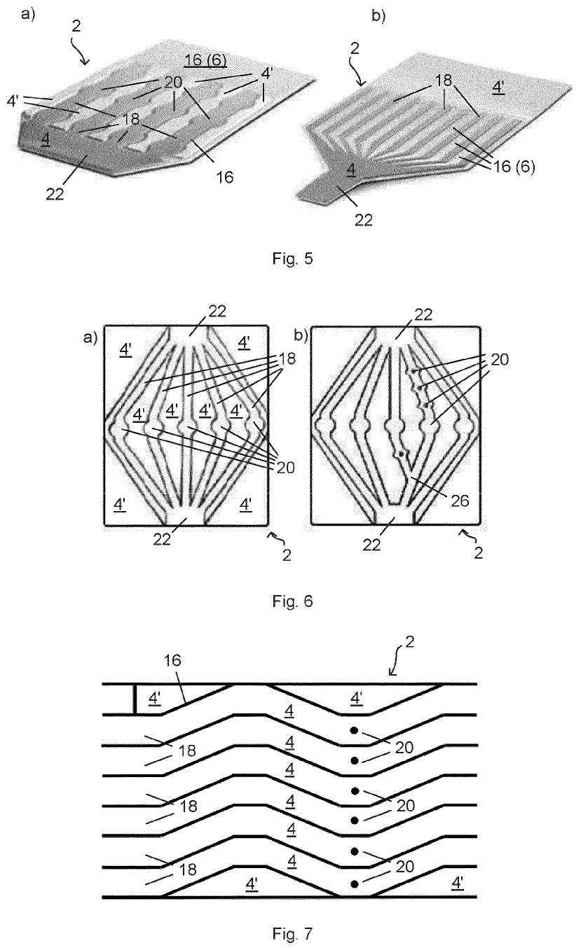

FIG. 5 shows membrane elements 2 released from a product 1 with exemplary shapes and/or structures.

At one end in the membrane layer 4, the structured membrane element 2 in FIG. 5 a) comprises an application zone 22, which is in particular suitable for applying a fluid to be tested and from which a plurality (e.g., four) of flow lines 18 with a plurality (e.g., three) of consecutive mixing and/or reaction zones 20 respectively extend, wherein the width of the flow lines 18 is smaller than the widths of the membrane element 2. The formation of such flow lines 18 increases concentration of the fluid on and/or along the flow lines 18 and the mixing and/or reaction zones 20, thus achieving an increased reaction intensity in these mixing and/or reaction zones 20.

Furthermore, the membrane element 2 in FIG. 5 a) has a structure 16 at the end opposite to the application zone 22, the structure 16 forming a part, for example about a fifth to about a quarter, of the surface of the membrane element 2. In particular, the membrane layer 4′ was ablated and/or removed in this structure 16, substantially over a surface. Such an adaptation is in particular advantageous in order to achieve improved handling of the membrane element 2. In particular, the membrane element 2 can be gripped at this end with tweezers without compromising the membrane layer 4 necessary to perform the test. The membrane element 2 can also be labeled in this area.

The application zone 22, the flow lines 18 and the mixing and/or reaction zones 20 are preferably formed and/or shaped by structures 16 and/or gaps 24.

In the example shown, the membrane element 2 in FIG. 5 a) preferably comprises a plurality of substantially isolated membrane layers 4′, which are substantially delimited and/or spaced apart from the application zone 22, the flow lines 18 and the mixing and/or reaction zones 20 such that a fluid applied in the application zone 22 can essentially not reach these isolated membrane layers 4′. In particular, these isolated membrane layers 4′ are arranged between the flow lines 18 and space them apart from one another. Such an embodiment of the membrane elements 2 is in particular advantageous because the processing effort for removing and/or cutting and/or melting the membrane layer 4, for example by an ablation device 14, is reduced as a result. In addition, the presence of one or more isolated membrane layers 4′ permits an optical reference with respect to the distribution of the fluid in the flow lines 18.

At one end in the membrane layer 4, the structured membrane element 2 according to FIG. 5 b) comprises an application zone 22 for applying a fluid, from which a plurality (in the example shown ten) of flow lines 18 extend. The flow lines 18 are substantially fan-shaped in a first region of the membrane element 2 and are arranged substantially parallel in relation to one another in a second region. Compared to the membrane element 2 according to FIG. 5 a), no isolated membrane layer 4′ is present between the flow lines 18 in the membrane element 2 according to FIG. 5 b). In other words: The structures 16 of the membrane element 2 of FIG. 5 b), which form the flow lines 18, extend over the entire distance of the respective flow lines 18 and space them apart from one another. Such an adaptation is in particular advantageous in order to achieve an improved delimitation of the flow lines 18 against one another.

The membrane element 2 of FIG. 5 b) further comprises an isolated membrane layer 4′ at the end of the membrane element 2 opposite the application zone 22. As with respect to the membrane element 2 of FIG. 5 a), this in particular facilitates improved handling of the membrane element 2.

However, reduced effort is required for producing the membrane element 2 of FIG. 5 b) since the insulated membrane layer 4′ is not ablated and/or removed.

As described in particular with regard to FIG. 3, the released membrane elements 2 of FIGS. 5 a) and 5 b) can at least regionally comprise adhesive on their (not shown) bottom, for example to allow an advantageous fixing of the membrane elements 2 in an analyzer.

FIG. 6 shows exemplary structures of membrane elements, wherein the structures each form a plurality (e.g., five or ten) of flow lines 18, each of which extends from application zones 22. The flow lines 18 each extend regionally substantially fan-shaped, i.e., they extend from the application zones 22 at different angles and/or spaced apart from one another, wherein the angles of the outer flow lines 18 (e.g., about 135°) are in particular greater than those of the central flow line (e.g., about 90°).

The membrane element 2 of FIG. 6 a) is formed substantially axis-symmetrically and comprises two opposite application zones 22 as well as several (e.g., five) mixing and/or reaction zones 20, which have a substantially circular shape and are arranged substantially centered between the mounting zones 22. The flow lines 18 extend substantially in a fan-shaped manner from respectively one application zone 22 to respectively one mixing and/or reaction zone 20. This embodiment is in particular advantageous if two fluids are applied to the membrane element 2 separately from one another and are mixed with one another and/or are to react with one another in a specific region of the membrane layer 4.

Contrary to the membrane element 2 of FIG. 6 a), the membrane element 6 b) is not symmetrical. The membrane element 2 of FIG. 6b) in particular comprises a single fork and/or branch 26 in a flow line 18.

The fork 26 divides a fluid applied to the lower application zone 22 and flowing along the corresponding flow line 18 into two flow lines 18 such that the fluid travels to the corresponding mixing and/or reaction zones 20 at a reduced concentration. A membrane element 2 can comprise a plurality of such and/or similar forks 26 for (re-)directing a fluid.

Furthermore, the membrane element 2 of FIG. 6 b) preferably forms one or more further mixing and/or reaction zones 20 along a flow line 18, into which DNA, RNA and/or proteins can preferably be introduced—for example by means of “dispensers” and/or “spotter”. This is in particular used to test the fluid before the latter reaches the mixing and/or reaction zones 20 arranged substantially centered between the application zones 22. The quality and/or concentration and/or other properties of the fluid can thus be tested and/or checked before the latter reaches the centrally arranged mixing and/or reaction zones 20.

The membrane elements 2 of FIGS. 6 a) and 6 b) further comprise substantially isolated membrane layers 4′ present in the corners and between the flow lines 18. Alternatively, the isolated membrane layers 4′ can be ablated, thus exposing the support layer 6 located underneath.

FIG. 7 shows a detail of an exemplary, structured membrane element 2 comprising a plurality (e.g., six) of flow lines 18 in the membrane layer 4. In this example, the flow lines 18 are arranged substantially parallel and extend substantially identically in a meander-shaped arrangement over the membrane element 2. Such an adaptation in particular improves the capillary effect of the flow lines 18 to advantageously conduct fluids along said flow lines.

Each of the flow lines 18 preferably comprises a mixing and/or reaction zone 20, in which for example DNA, RNA, and/or proteins were introduced.

When a fluid to be tested reaches a mixing and/or reaction zone 20 comprising DNA, RNA and/or proteins, said mixture can react with the DNA, RNA, and/or protein. The reaction preferably results in a visible change in the color in at least a part of the mixing and/or reaction zone 20, which can in particular be detected with an analyzer. If no reaction occurs, the fluid to be tested probably has no or too low a concentration of the substance required for the reaction, such as certain antibodies.

The flow lines 18 of the membrane element 2 of FIG. 7 are formed by structures 16, preferably by removing at least a portion of the thickness of the membrane layer 4. Alternatively and/or additionally, the flow lines 18 can be formed at least partially by forming a gap 24 in the membrane layer 4 and the support layer 6. The membrane element 2 further comprises a plurality (e.g., 4) of isolated membrane layers 4′, which are not reached by a fluid guided by the flow lines 18. The isolated membrane layers 4′ are particular intended for improved handling and/or an increased shape stability of the membrane element 2.

### LIST OF REFERENCE NUMERALS

1 Product for providing membrane elements
2 Membrane element
4 Membrane layer
4′ Isolated membrane layer of a membrane element
4″ Membrane layer not comprised by any membrane element
6 Support layer
6″ Support layer not comprised by any membrane element
8 Carrier film
10 Adhesive
12 Adhesive device
14 Ablation device
16 Structure
18 Flow line
20 Mixing and/or reaction zone
22 Application zone
24 Gap
26 Fork

1. A product for providing membrane elements comprising:
a carrier film; and
a membrane film at least comprising:
a support layer; and
a membrane layer;
wherein the membrane film is releasably coupled to the carrier film; and
wherein the membrane film comprises a plurality of substantially discrete membrane elements.

2. The product according to claim 1, wherein the support layer is arranged between the carrier film and the membrane layer.

3. The product according to claim 1, wherein the membrane layer comprises a porous, absorbent material; and/or
wherein the membrane layer comprises:
cellulose nitrate and/or cellulose acetate, and/or polysulfone, in particular polyethersulfone, PESU.

4. The product according to claim 1, wherein the membrane layer has a thickness of about 50 μm to about 200 μm and/or
wherein the carrier film comprises a carrier material having a silicone coating.

5. The product according to claim 1, wherein the carrier film has a thickness of approximately 100 μm to approximately 300 μm; and/or
wherein the support layer has a thickness of about 10 μm to about 20 μm.

6. The product according to claim 1, wherein the support layer of the membrane film is releasably coupled to the carrier film by means of adhesive and/or
wherein the membrane layer of the membrane film is coupled to the support layer of the membrane film by means of adhesive.

7. The product according to claim 6, wherein the adhesive comprises a contact adhesive and/or a melt adhesive.

8. The product according to claim 6, wherein the adhesive has a layer thickness of about 8 μm to about 20 μm.

9. The product according to claim 1, wherein the membrane elements have an at least partially structured surface.

10. The product according to claim 1, wherein the membrane elements have one or more structures in the membrane layer.

11. The product according to claim 10, wherein the one or more structures in the membrane layer were formed by at least partial ablation of the membrane layer; and/or
wherein a structure forms one or more flow lines for guiding a fluid and/or controlling a fluid flow; and/or
wherein a structure forms one or more mixing and/or reaction zones.

12. A method for producing a product for providing membrane elements, comprising the steps:
providing a support layer;
releasably coupling a carrier film to a first side of the support layer;
coupling a membrane layer to a second side of the support layer;
forming a plurality of membrane elements by at least partial ablation of the support layer and the membrane layer.

13. The method according to claim 12, wherein the membrane elements are formed and/or structured by means of laser cutting.

14. The method according to claim 12, further comprising the step:
applying an adhesive to the support layer and/or to the membrane layer to couple the support layer to the membrane layer; and/or
applying an adhesive to the support layer and/or the carrier film to releasably couple the support layer to the carrier film.
